(19) United States
(12) Patent Application Publication
(10) Pub. No.: US 2016/0205897 A1
(43) Pub. Date: Jul. 21, 2016

(54) CENTRAL ABSORPTION PET URINAL PAD

(71) Applicant: Jiangsu Zhongheng Pet Articles Joint-Stock Co., Ltd., Jiangsu (CN)

(72) Inventor: Qiu Bin, Jiangsu (CN)

(73) Assignee: Jiangsu Zhongheng Pet Articles Joint-Stock Co., Ltd., Jiangsu (CN)

(21) Appl. No.: 14/996,113

(22) Filed: Jan. 14, 2016

(30) Foreign Application Priority Data
Jan. 15, 2015 (CN) 201520029476.8

(51) Int. Cl. A01K 23/00; A61F 13/53

(52) U.S. Cl. CPC A01K 23/00; A61F 13/53; A61F 2013/530131; A61F 2013/15186

(57) ABSTRACT

A central absorption pet urinal pad has a stratiform structure, which includes: a non-woven fabric coating layer on a top side; a plastic film leakage prevention layer on a bottom side; an absorbing layer arranged between the non-woven fabric coating layer and the plastic film leakage prevention layer, wherein the absorbing layer is smaller in dimensions such that a platform structure is formed above the absorbing layer in a center region of the central absorption pet urinal pad; and a toilet paper absorbing layer arranged between the plastic film leakage prevention layer and the absorbing layer.

### CROSS-REFERENCE TO RELATED APPLICATIONS

This Application claims the priority of Chinese Patent Application No. CN201520029476.8, filed on Jan. 15, 2015, the disclosure of which is incorporated by referecne in its entirety.

### TECHNICAL FIELD

The invention relates to a pet urinal pad, and more particularly, to a central absorption pet urinal pad, which belongs to the technical field of pet products.

### BACKGROUND ART

Currently, with the improvement of people's living standards, pets, such as cats, dogs or the like, have entered the lives of people, playing an increasingly important role. People are also demanding more for the sanitation of their pets. However, in daily life, it is very troublesome to dispose of pet excreta, which require the pet masters to pay much attention. To protect the environments and also to create a more comfortable habitat for the pets to meet the animal right demands for the pets, and at the same time to reduce the tedious work for people sweeping the pet excreta, quite a number of people have use pet urinal pads while keeping pets.

An existing pet urinal pad typically includes a non-woven fabric surface coating layer. An absorbing layer, formed by mixing of wood pulp, PP fibers and super absorbent molecules, is arranged below the non-woven fabric surface coating layer, and then a PE film bottom leakage prevention layer is arranged on the external side of the absorbing layer. However, such a pet urinal pad, while in use, cannot absorb urine in time in case of a large volume pet urine, such that the urine leaks out from the edges of the urinal pad. Therefore, to prevent side leakage, the urinal pads available in the market are getting bigger and bigger. In this way, although the side leakage problem is solved, the following problems are still existing: firstly, excessive materials are wasted; secondly, although the urinal pad becomes bigger, the pets will not urinate and defecate in the center position, and side leakage may still occur; and thirdly, when the pets urinate and defecate on the urinal pad, it is inevitable to bring the urine out when the pets move due to the larger absorption area.

### SUMMARY OF THE INVENTION

An object of the invention is to provide a central absorption pet urinal pad so as to solve the foregoing problems in the prior art.

To solve the foregoing technical problem, the invention employs a technical scheme as follows:

A central absorption pet urinal pad, wherein a urinal pad body has a stratiform (layered) structure, which includes: a non-woven fabric coating layer on the top side, and a PE film leakage prevention layer on the bottom side; wherein an absorbing layer is arranged in a middle position between the non-woven fabric coating layer and the PE film leakage prevention layer; a lug boss is formed on the position of the absorbing layer of the urinal pad body; and a toilet paper absorbing layer is also arranged between the leakage prevention layer and the absorbing layer.

The outer edge of the absorbing layer and the outer edge of the toilet paper absorbing layer are overlapped.

For better absorption, a plurality of absorption holes are uniformly arranged on the coating layer on the position of the absorbing layer.

The invention has the following advantageous effects that: through the bulging of the absorbing layer, the middle part of the entire urinal pad is set as a lug boss structure, so that the volume of the urinal pad body may be reduced and materials are saved on one hand, and on the hand, the lug boss may enable pets to distinguish a position for urinating and defecating so that the pets may urinate and defecate on the lug boss, which prevents side leakage and improves the utilization factor.

### BRIEF DESCRIPTION OF THE DRAWINGS

FIG. 1 shows a structure diagram showing a longitudinal cross section of a pet urinal pad in accordance with an embodiment of the invention.

FIG. 2 shows a top view of a pet urinal pad, illustrating absorption holes on the top layer, in accordance with an embodiment of the invention.

In the figures: 1 refers to a coating layer, 2 refers to an absorbing layer, 3 refers to a toilet paper absorbing layer, and 4 refers to a leakage prevention layer.

### DETAILED DESCRIPTION

The technical characteristics of the invention will be described in details hereinafter with references to the drawings and embodiments.

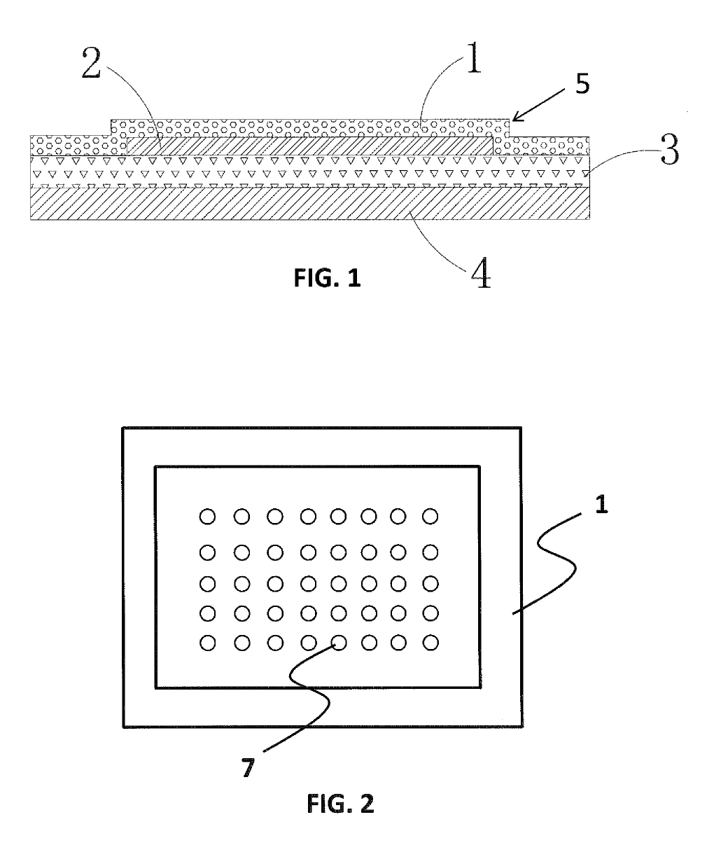

According to embodimnents of the invention, a central absorption pet urinal pad is shown in FIG. 1. As shown, a urinal pad body has a stratiform (layered) structure, which includes: a non-woven fabric coating layer 1 on the top side and a plastic (e.g., PE or PP) film leakage prevention layer 4 on the bottom side; wherein an absorbing layer 2 is arranged between the non-woven fabric coating layer 1 and the PE film leakage prevention layer 4; wherein the absorbing layer 2 is smaller in dimensions than the non-woven fabric coating layer 1 such that a raised platform (boss) structure 5 is formed above the absorbing layer 2 of the urinal pad body; and a toilet paper absorbing layer 3 is also arranged between the leakage prevention layer 4 and the absorbing layer 2.

In accordance with some embodiments of the invention, the outer edge of the absorbing layer 2 and the outer edge of the toilet paper absorbing layer 3 may align—i.e., both the absorbing layer 2 and the toilet paper absorbing layer 3 may be of a smaller dimenstion, as compared with the non-woven fabric coating layer 1 and the film leakage prevention layer 4. In these embodiments, the raised platform structure may be supported by both the absorbing layer 2 and the toilet paper absorbing layer 3.

In accordance with some embodiments of the invention, the absorbing layer 2 may be formed by mixing of wood pulp, PP fiber and/or fast absorption super absorbent molecules.

For better absorption, a plurality of absorption holes 7 may be arranged on the non-woven fabric coating layer 1 at locations above the absorbing layer 2 (FIG. 2). In preferred embodiments, the absorption holes 7 are uniformly arranged in the non-woven fabric coating layer 1. The absorption holes may be cutouts in the non-woven fabric coating layer 1 or may be made of a material more water permeable than the non-woven fabric.

When in use, the platform (boss) structure of the central absorbing layer 2 enables the pets to recognize locations for urinating and defecating, so that the pets may urinate and defecate on the raised platform easily, which prevents side leakage and improves the utilization factor.

What is claimed is:

1. A central absorption pet urinal pad having a stratiform structure, comprising:
a non-woven fabric coating layer on a top side;
a plastic film leakage prevention layer on a bottom side;
an absorbing layer arranged between the non-woven fabric coating layer and the plastic film leakage prevention layer, wherein the absorbing layer is smaller in dimensions such that a platform structure is formed above the absorbing layer in a center region of the central absorption pet urinal pad; and
a toilet paper absorbing layer arranged between the plastic film leakage prevention layer and the absorbing layer.

2. The central absorption pet urinal pad according to claim 1, wherein an outer edge of the absorbing layer and an outer edge of the toilet paper absorbing layer are aligned.

3. The central absorption pet urinal pad according to claim 1, wherein a plurality of absorption holes are arranged on the non-woven fabric coating layer above the absorbing layer.
